(19) United States
(12) Patent Application Publication
(10) Pub. No.: US 2019/0341128 A1
(43) Pub. Date: Nov. 7, 2019

(54) SECURE SYSTEM FOR COORDINATING AND PROVIDING BLOOD SAMPLE ANALYSIS

(71) Applicant: NEOMEDA LLC, Jersey City, NJ (US)

(72) Inventor: Dennis Yurovsky, Jersey City, NJ (US)

(21) Appl. No.: 16/403,426

(22) Filed: May 3, 2019

Related U.S. Application Data
(60) Provisional application No. 62/666,537, filed on May 3, 2018.

(51) Int. Cl. G16H 10/40; G16H 10/60; G06F 11/36

(52) U.S. Cl. CPC G16H 10/40; G16H 50/20; G06F 11/3672; G16H 10/60

(57) ABSTRACT

Systems for coordinating and providing blood sample analysis are described herein. The system comprises a web server implemented as a computer system in communication with a computer network. The system is configured to perform certain operations in response to messages received from devices over the Internet. The system comprises interfaces (e.g., communication ports and supporting software) for communication with client devices external to the server. The system is configured to receive an order for a blood sample an analysis. To that end, the web server is in communication with a physician's client computer device via a communication network. The system implements computers and equipment that receive requests for blood tests and provide related services including, for example, automated and secure testing and reports of tests.

### CROSS-REFERENCE TO RELATED APPLICATIONS

This application claims the benefit of U.S. Provisional Patent Application No. 62/666,537, filed May 3, 2018, which is hereby incorporated herein in its entirety.

### BACKGROUND

The present application relates to systems and methods for automated biological testing systems and in particular to automated systems for performing testing and analysis on blood samples.

### SUMMARY OF THE INVENTION

In accordance with principles of the invention, systems, methods, computer readable medium for securely providing and processing biological specimen analysis are contemplated. For example, a system is contemplated comprising, a web server configured to receive an order for a biological specimen analysis from a provider client computer, a patient database computer, in communication with the web server and patient database, configured to receive the order and generate order parameter data and a unique ID associated with the order, and store data comprising the order parameter data, the unique order ID, and test results for each order in the patient database as a patient database record, lab test equipment configured to analyze biological specimens and transmit raw test data, a lab database computer in communication with the lab test equipment and comprising a lab database configured to store the raw test data received from the lab test equipment, and a translator computer in communication with both the patient database computer and the lab database computer and configured to detect storage of the order in the patient database, and further configured to communicate with the lab database computer to create a placeholder record for the order in the lab database, the placeholder record being associated with the unique order ID and comprising the order parameter data, and further comprising cells for storing the raw test data received from the lab test equipment and associated with the order.

In some embodiments, the translator computer communicates with the lab database computer to monitor the lab database for receipt of the raw test data transmitted by the lab test equipment, and wherein when the translator computer detects the receipt of the raw test data, the translator computer retrieves the raw test data from the lab database, performs an analysis on the raw test data to generate test results, and transmit the test result to the patient database computer for storage in the patient database record of the unique order ID.

In some embodiments, the patient database computer is further configured, in response to receiving an order, to transmit a notification of an available order to a pool of phlebotomist devices based on location or other criteria, designate a phlebotomist device to be matched to the order in response to that device accepting the order, track the phlebotomist device within a predetermined time period before the appointment, and coordinate communications between the phlebotomist and the patient.

In some embodiments, the analysis performed by the translator computer is performed according to the order parameters associated with the unique order ID and comprises translating the raw test data to a human readable format.

In some embodiments, the analysis performed by the translator computer detects abnormal results and provides a diagnostic.

In some embodiments, the lab test equipment comprises a scanner configured to scan the unique order ID on the test vial and to associate the raw test data generated from testing the test vial with the unique order ID of the test vial for transmission to the database computer.

In some embodiments, the translator computer monitors the lab database in real time.

In some embodiments, the system further comprises a phlebotomist computer device in communication with the web server and configured to receive the order and the unique order ID from the web server, wherein the web serve retrieves the order and the unique order ID from the patient database, the phlebotomist computer device being further configured to enable printing of the unique order ID as a label comprising the unique order ID and a barcode for the unique order ID.

In some embodiments, the system further comprises a patient computer device in communication with the web server and configured to receive the test results from the web server, wherein the web server retrieves the test results from the patient database.

In some embodiments, the order parameter data comprises at least one of instructions to perform a specific test and instructions for translating the raw test data.

In some embodiments, the lab test equipment retrieves the order parameter data from the database computer and performs testing according to the order parameter data.

### BRIEF DESCRIPTION OF DRAWINGS

The nature and various advantages of the present invention (and other inventive aspects) will become more apparent upon consideration of the following detailed description, taken in conjunction with the accompanying drawings, in which like reference characters refer to like parts throughout, and in which:

FIG. 1A depicts a block diagram of an illustrative system in accordance with some embodiments of the invention;

FIG. 1B depicts a flow chart of an illustrative process in accordance with some embodiments of the invention;

FIG. 1C depicts a functional block diagram of an illustrative system in accordance with some embodiments of the invention;

FIGS. 2A-2B, 3A-3B, and 4A-E depict illustrative display screens as part of user interface features and related functionality in accordance with some embodiments of the invention;

FIG. 5 depicts a functional block diagram of an illustrative system in accordance with some embodiments of the invention;

FIGS. 6A-6B depict illustrative display screens as part of user interface features and related functionality in accordance with some embodiments of the invention, and

FIG. 7 depicts a functional block diagram of an illustrative system in accordance with some embodiments of the invention.

### DETAILED DESCRIPTION

In accordance with principles of the present invention, embodiments are provided that improve over known prior art in different ways. One objective of embodiments of the present invention relates to providing an online automated clinical diagnostic system that is “live” and available around the clock and is also highly secure from a network and operational perspective.

For example, in implementation, a diagnostic test equipment that is adapted for performing automated high volume testing of biological samples is provided as part of the system. Such high volume test equipment for performing blood testing and analysis is currently available on the market but vendors such as Beckman Coulter. The diagnostic test equipment is, for example, adapted to handle 2000 tests per hour (such for conducting basic chemistry blood tests), the throughput can depend on the particular test. The testing and analysis typically includes the following tests comprehensive chemistry screen, liver function, electroyltes, or lipids testing. The diagnostic test equipment includes a physical receptacle such as a tray (e.g., removable tray) for receiving vials of biological samples for testing. The diagnostic test equipment further includes a scanner or other mechanism that automatically reads the labels on vials such as machine readable code such as a bar code or other forms of tags (e.g., the equipment is configured to automatically read human readable information). Once one or more vials are loaded (e.g., using a tray), the equipment automatically performs tests and generates results from the testing and analysis (i.e., without human intervention or requiring human involvement). The equipment also includes a computer and one or more communications ports. The equipment is configured to communicate with a computer, typically an external database or an external database server (having a database). The equipment and computer are configured to be in a secure relationship. This is implemented by way of direct wired cable connection between the equipment, through a directed device to device connection using a cable, a network connection in a secure room or location, or by implementing a trusted relationship using a security protection such as by using encryption (encryption keys). The diagnostic test equipment generates values representing individual results of tests performed on blood (or other biological samples) and transmits the values to the database server. The database server saves the values in the database. The database or database server may be considered to be part of the diagnostic test equipment (for example because, it is dedicated to supporting the test equipment and is running a software application that is configured specifically to interface with the test equipment).

Embodiments of the present invention provide improved security by for example, isolating or insulating the diagnostic test equipment from external networks such as the Internet by electronically (e.g. from a network perspective) hiding the diagnostic test equipment behind multiple layers of electronic barriers. For example, a second database server is implemented in the system. The second database server is configured to have an unconventional interrelationship with the above-mentioned database server (the database server supporting the test equipment), which will be referred to as the first database server for convenience. A software module or application is implemented on the first database server. The software is sometimes referred to as a monitoring application. This software operates “invisible” to the interoperation of the first database server and the diagnostic test equipment. It is configured to be able to view the operations that are performed in the database of the first database server. It can spy on or peek into the first database. For example, the first database server and its database may be operating using a proprietary software application and related proprietary database structures. The proprietary software application manages the relationship with the test equipment (via its data connection) and stores generated values. The first database server is for example an Oracle database. The monitoring application is separately installed on the first database server (or on its database) to be able to view and read activity in the first database. Software applications can be integrated into the system to provide the monitoring application. The application provides for synchronization and monitoring services.

The second database server uses the monitoring application to read data values from locations in the first database and saves the data values that are read into corresponding database fields in the second database. The monitoring application may have a portion that is running on the second database server or may have interface to an application running on the second database server for carrying its functionality. The monitoring application is adapted to only read data values that are stored in the first database and is without the ability to write to the first database. The monitoring application is preferably monitoring whether a new activity has occurred in the first database on a real time and continuous basis. In other words, as soon as a new value is saved, the monitoring application sees the activity in the first database, copies the value, and saves that new value into the second database in a corresponding field (e.g., in cooperation with the second database server, or an application on that server). For example, in a simple example, the value for blood type is copied into a field for blood type (for that order) in the second database.

The second database server is connected to a web server (e.g., an HTTP web server) and supports the operation of the web server with client devices over the Internet. The web server is an Internet facing element and is proxy or firewall for the second database server. The web server can for example receive a request for test testing in connection with a particular order. The web server transmits the request to the second database server. The second database server can access the second database and generate a response message containing the data values (fi they exit yet) from the second database that responds to the request from the web server. In this arrangement, the test equipment and the first database server (and database) are topologically hidden behind the first database server and the web server (in relation to computers on the Internet) and are also topologically hidden from the first database server and the web server.

This arrangement improves the security applied to the equipment and the first database because it avoids network discovery and unauthorized access. It is very important to maintain the privacy and accuracy of the test data and the test equipment. Hacking or overloading of those systems can have negative consequences. This arrangement provides a one way data path (data values copied and stored from first database to second database) and it can do so without relying on a communications protocol or networking protocol, a protocol that defines a messaging structures and signaling between communications end points).

One related issue is that this one way data path does not allow for automated creation of new database records. For example, the second database will mirror or substantially mirror the data values and fields in the first database but there has to be a method to implement this arrangement. A process is implemented in which the first database server and first database are configured to include a designated folder. The designated folder is preferably distinct and separate from the first database and the designate folder does not provide access to the values or records in the first database. The designated folder is designated as a location in which a file of a particular type can be downloaded that notifies the first database server that a new record and corresponding data fields are to be created. For example, when a new order is received that requests testing and phlebotomist services, the second database server receives the message from the web server. The second database server determines that it is a new request and in response, creates a file (e.g., an HL7 file), and downloads the file into the designated folder. The file contains the information or instructions that define to the first database server the necessary operation to create a new record in the first database that corresponds to the new request. A similar record is created in the second database (e.g., using a different type of database such as SQL and Oracle in the first database). The file, when downloaded, can also include the information specifying the tests that are to be performed.

This operation again limits the communications and insulates the first database from the risk of being accessed or hacked (which of occurred, could risk changing results or jeopardizing operations).

This arrangement also improves the volume of test and reports that are generated because it removes the involvement of human technicians from the testing and operation of the system.

Preferably, the test equipment and first database server are not connected to or on the same network from the web server and second database server. In other words, the network communications hardware and software on the test equipment and first database server do not have a physical path (cables, routers, etc.) or wireless connection to the web server and second database server. For example, the components are configured through hardware or software to be without the ability to execute communications in accordance with a network communications or device communications protocol such as standard protocols used in implementing the OSI layers to communicate with each other (e.g., by using network addresses). The only available communication between the web server/second database server and test equipment/first database server is for example the “peeking,” copying and saving of data (using the monitoring application, which can be part of a larger software such as a translator, mentioned below) and the downloading of a file in a designated folder).

From a system-wide perspective, embodiments of the present invention disclose a system for coordinating and providing blood sample analysis. The system provides for the ordering of blood sample testing from a health care provider's computer device, such as a desktop computer at the doctor's office or a mobile device operated by a healthcare facility staff. The system further allows the patient to be matched with a phlebotomist to arrange an appointment to collect blood. The system causes blood sample vials bearing unique ID labels to be shipped to the phlebotomist for use to collect the patient's blood. The system tracks the arrival of blood sample vials to a participating lab. The lab may include test equipment, preferably automated high volume test equipment mentioned above, which may for convenience by also referred to as test equipment, in communication with a lab database computer (referred to above as first database server) of the system. The system uses the provider's order to direct the test equipment to perform analysis of the blood sample in accordance with the parameters of the order. The test equipment is configured to communicate automatically raw test data directly to the system. The system then translates the raw test data into a human readable report that may comprise diagnostics specified in the order. The report may be transmitted to the provider and patient client computer devices via a network.

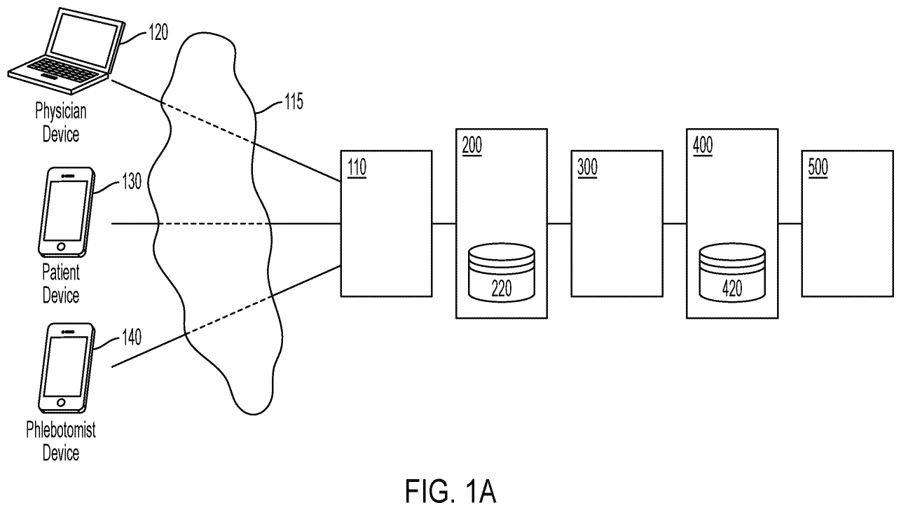

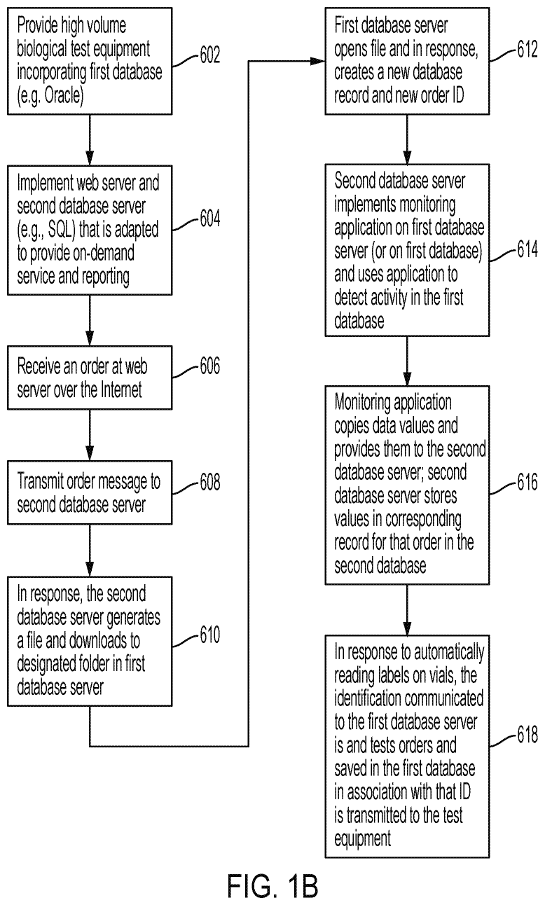

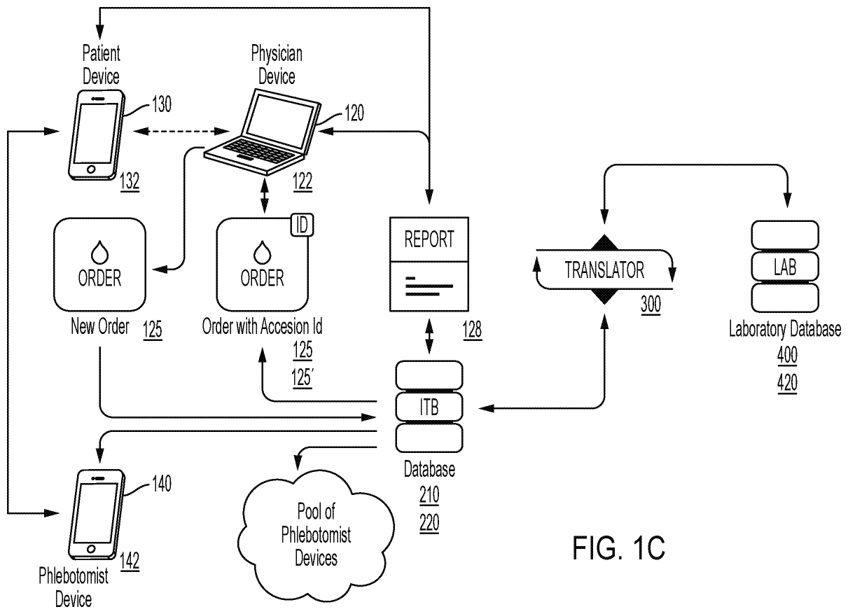

FIG. 1A illustrates a diagram of the system for coordinating and providing blood sample analysis, according to an embodiment of the principles described herein. The system 100 comprises a web server 110. The web server 110 may be implemented as a computer system in communication with a computer network, and that stores and delivers websites or web pages upon request to client computer devices via the network. A web server is a specialized computer (software/hardware combination implemented on a computer) that is known to those ordinary skill in the art. As part of its functionality, it is configured to perform certain operations in response to messages received from devices over the Internet. The server 110 comprises interfaces (e.g., communication ports and supporting software) for communication with client devices external to the server. The interfaces may be implemented in hardware to send and receive signals in a variety of mediums such as copper, optical, and wireless. The signals may be transmitted or received using a variety of different protocols including non-transitory ones. The server 110 can include more than one server. The server 100 is configured to receive an order for a blood sample an analysis. To that end, the web server is in communication with a physician's client computer device 120 via a communication network 115.

The communication network 115 can include the Internet, a cellular network, a telephone network, a computer network, a packet switching network, a line switching network, a local area network (LAN), a wide area network (WAN), a global area network, or any number of private networks currently referred to as an Intranet, and/or any other network or combination of networks that can accommodate data communication. Such networks may be implemented with any number of hardware and software components, transmission media and network protocols. Although FIG. 1 represents the network 115 as a single network, the network 115 can include multiple interconnected networks listed above.

The physician client computer device or physician device 120 can be any platform capable of computations and of communication across a network 115. Non-limiting examples may include a computer, laptop, tablet, mobile device, server, or any other electronic device capable of communication across a network of computers. The physician client device 120 is configured with one or more processors that process instructions and run software that may be stored in memory. The processor also communicates with the memory and interfaces to communicate with other devices. The processor can be any applicable processor such as a system-on-a-chip that combines a CPU, an application processor, and flash memory. The physician device 120 is provided with a display device, and includes a variety of user interfaces such as a keyboard, a touch screen, a trackball, a touch pad, and/or a mouse.

The physician client device 120 is operated by medical staff such as a physician, nurse, or any other medical specialist or staff. The physician client device 120 may be provided with an application or physician portal to enable the ordering and management of blood sample analysis using system 100. The application or physician portal is in communication with web server 100. The physician device 120 may also be provided with a web browser for accessing a physician web portal 122 served as a web page or web pages by web server 110. The web portal 122 allows medical staff to order and manage the blood sample analysis process. The portal 122 enables medical staff to register on the system as a healthcare provider. Initially, the portal 122 may provide for both practice registration, and registration for individuals in a health care provider practice such as physicians, nurses, or other medical professional. The provider information is securely transmitted to the system via the network for verification, validation, and approval of the provider's registration. Upon approval, the provider is registered with the system and the now-registered users are supplied with login credentials for use with the portal. A registered medical professional with the appropriate credential may log into their account to order blood sample analysis for patients, manage such orders, and review test results provided by the system after fulfillment of the orders.

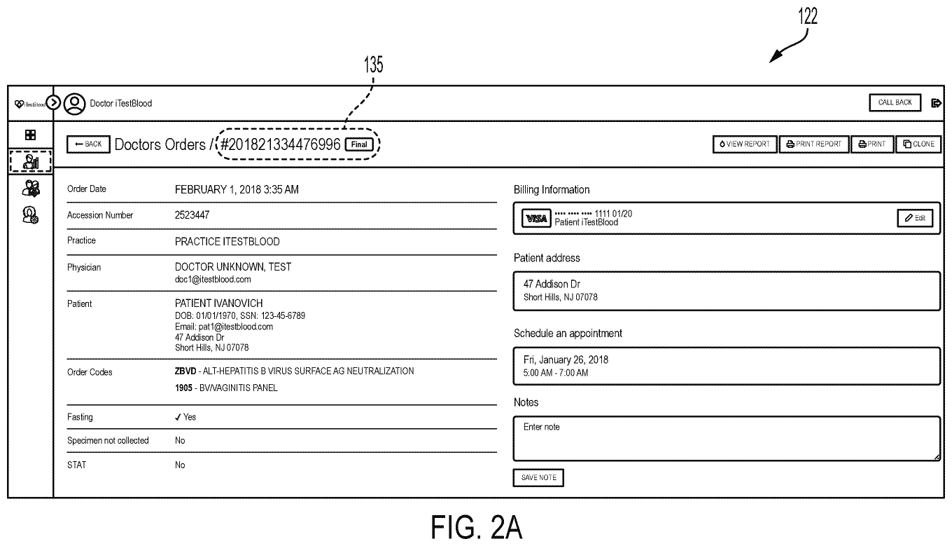

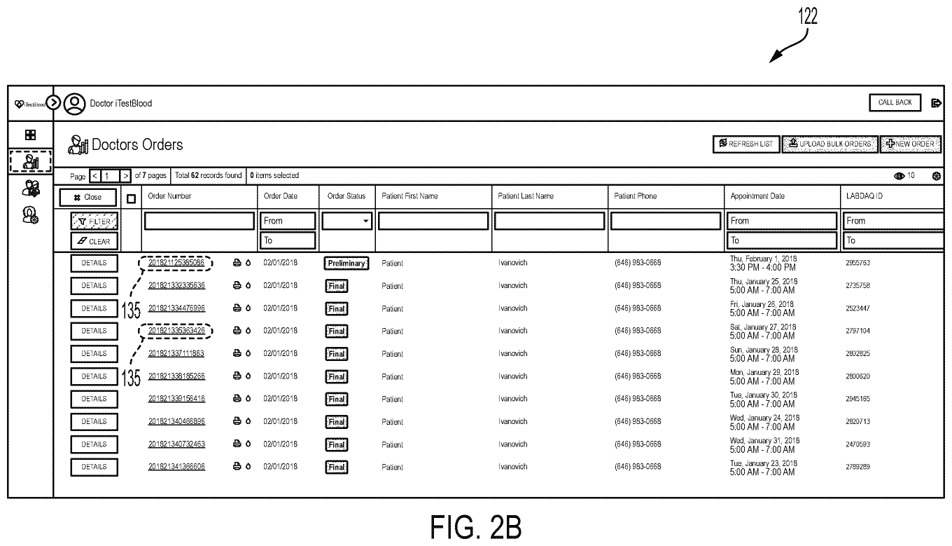

FIG. 2A-2B illustrate screenshots of an exemplary physician portal 122, according to an embodiment of the present invention. Specifically, the FIG. 2A illustrates exemplary elements of an order for a blood test. After the physician longs into the portal 122 and initiates an order, the physician is prompted by the portal to enter a variety of information pertaining to the order. After the order information is entered, an order summary may be displayed, as illustrated in FIG. 2A. The displayed order summary includes information such as the order date, physician information, patient information (e.g., name, address, etc.), and billing information (credit card number, etc.). In particular, the order summary includes an accession number 125′ generated by the system when the order was placed. The accession number 125′ is a unique identifier for a blood sample vial to be provided by the patient. The accession number 125′ is generated at the time of the order placement by the lab database server when the record for that order is created in that database, and serves to track the order and associated vials throughout the lifecycle of the order, i.e., from the order placement to the provision of results to the physician and patient. In some embodiments, such as illustrated, the accession number is the order number. This is also sometimes referred to as a unique identification or identification and it does not need to be limited to purely numbers. The order summary further includes the order parameters, i.e., the information specific to the blood test to be performed. The order parameters include the desired test(s) to the performed on the patient's blood sample and the parameters of such tests. The tests may be selected from a menu of tests, or entered and displayed as codes consistent with the standards of the industry or practice. A physician can manage more than one order though the portal 122, and the portal allows users the opportunity to view a list of pending orders, as illustrated in FIG. 2B.

Referring back to FIG. 1, after the blood sample order is entered on the physician client computer 120, the order is transmitted over the communication network 115 to web server 110. The web server 110 is configured to receive and process orders for blood sample analysis from client computer devices such as described above. As will be further described below, the web server 110 serves as a communication hub for client devices outside of the system 100. In particular, the web server provides the interface between external devices such as the physician client device and the rest of system 100.

The web server 110 transmits the order comprising order parameters to a patient database computer 200. The web server 110 may be in communication with the patient database computer 200 over a network or via a direct communication link. The patient database computer is sometimes referred to as the second database server. The patient database computer 200 is configured to process and store the order for blood sample analysis received from the web server 110. During processing, the patient database computer generates the accession number or unique identifier for the patient. The accession number is included with the patient's record and associated therewith. The patient database computer 200 comprises a patient database 220 for storing and managing orders received from physician client computer 120. In some embodiments, the database 220 may be a SQL database. The order may be stored in the patient database 220 as a record identified by the accession number and comprising the order parameter data. Further, additional storage is created in the patient record for storing test results or reports and interpretations thereof, as well all any other relevant information associated with the patient. After the accession number generated and associated with the order and the order parameters are stored, the web server 110 may retrieve the order from the patient database 220 for transmission to the physician client computer 120 to generate an order confirmation screen for the medical staff. An exemplary order confirmation screen is illustrated in FIG. 2A.

Staying with FIG. 1A, the system further comprises a translator 300. The translator 300 is in communication with the patient database computer 200. In some embodiments, the translator 300 may be a software module or application running on the database computer system 200. In some embodiments, the translator 300 comprise a separate translator computer system in communication with the patient computer 200 and database 220. The translator 300 manages the transfer of blood sample order parameters between the database computer 200 and the testing lab's computer systems and test equipment. The translator 300 further serves to receive, translate, and store the lab test results from the lab's computer systems 400 and test equipment 500 and into the patient database 220 for delivery to the physician client device 120 and patient client device 130, as will be explained further in this specification.

The system further comprises a second database computer 400 or lab database computer 400. The lab database computer 400 is in communication with the translator 300, from which it receives translations of order parameters stored in the patient database system 200. The lab database system 400 may be in communication with the translator 300 over a network or with a wired direct connection. The lab database computer 400 is also in communication with the lab equipment or test equipment 500 that is configured to perform tests on patients' blood samples. The lab database computer 400 comprises a database for storing test directives received from the translator 300 for the test equipment 500.

When the physician or other medical staff places an order for a blood test on the physician client computer 120 as described above, the order is stored in the database 220 of the patient database computer 200. The creation of a new test record in the patient database 220 is communicated to the translator 300. The translator 300 accesses and reads the order record in the patient database 220 via the communication link between the translator 300 and the patient database computer 200. The translator 300 then signals to the lab database computer 400 to create a corresponding test record for the same patient in the lab database 420. The test record in the lab database 420 may comprise directives for the lab equipment as communicated by the translator 300. For example, the directives may specify which particular equipment should perform a test on a blood sample vial carrying the patient's accession number 125′. The directives may further specify the test that the equipment may perform, and the parameters of the particular test. The directives may instruct the lab equipment to record certain information and discard others, for example. The test record in the lab database 420 thus comprises the instructions necessary to operate the test equipment for a particular patient.

To preserve the confidentiality of patient information, the translator 300 may not communicate the patient's name, address, or any other information sufficient to identify the patient, to the lab database computer 400. Instead, the test record created in the database computer is identified by the accession number 125′ generated by the patient database computer 200 and associated with the patient. Accordingly, any transaction and operation on the lab-side of the translator 300 occurs without the possibility of identifying the patient. The system 100 thus provides a wall between the patient's information, and the testing directives and results. These elements can only be reconciled on the patient/physician side of the translator 300.

The test record created in the lab database 420 further serves as a placeholder record for the test results transmitted by the test equipment 500 to the lab database. Before the tests are conducted and the results transmitted to the database computer 400, a placeholder record or empty database cell intended to hold the raw test data is also generated under the test record for the particular accession number 125′. The placeholder record serves to receive and store the test results from the test equipment 500. To assure the integrity of the system 100 against errors and tampering, the lab-side database 400 is configured to accept raw test data from the test equipment 500 only. Various hardware and software schemes may be provided to ensure exclusive write-access to the test record data in the lab-site database by the test equipment 500. In some embodiments, the system 100 is configured such that the translator 300 is unable to write to the lab database 420. Rather, the translator 300 is only able to communicate test directives to the lab database computer 400, which creates and saves them under the relevant record as identified by the accession number 125′. Accordingly, only the test equipment 500 can communicate test results to the lab database computer 400.

Referring back to FIG. 1A, after the patient database computer 200 stores the order in the patient database 220, the web server 110 generates a notification for a patient client device 130 to alert the patient of the entry of the order. The patient client computing device 130 may be a computer, tablet, or mobile device configured to connect to and communicate across a network 115. In some embodiments, the patient client computing device 130 may be a mobile device. The notification is transmitted over network 115 and may be received by a patient-side application or patient portal 132 running on the patient client computing device 130. Concurrently or alternatively, the web server 110 may provide access to the patient's order record in the patient database computer system 200. For example, a browser on the patient client computing device 130 may be used to open a web page provided by the web server 110 to display the order.

Before the patient portal 132 can be used on the patient display device, the patient must register with the system. In most respects, the registration and authentication process is similar to the previously-described medical staff registration and authentication process on the physician client computer 120. In particular, the patient may initially register his or her information with the system via the network and web server 110, and be provided with login credentials. Subsequently, the patient may use the login credentials to authenticate a session on the portal access with an application or a web browser.

With reference now to FIG. 1B, an illustrative process (and related system) can be implemented that can provide benefits such as improved security, reduced processing load, and improved efficiency. At step 602, a high volume biological test equipment is provided that incorporates a first type of database and database server (first database server) such as an Oracle database. At step 604, a web server and a second type of database server (second database server) is implemented that is adapted, among other things, to provide on-demand services and transmit information (e.g., test results) originating from the test equipment.

At step 606, the web server receives an electronic order over the Internet from a device such as a mobile device or a computer in a Doctor's office. At step 608, the web server transmits the new order (or a message representative of it) to the second database server. At step 610, in response to receiving the request, the second database server generates a setup file (setup new order file) and downloads the setup file to a designated folder in the first database or first database server, wherein the folder is designated and configured as a secure buffer area for receiving such files. At step 612, the first database server opens the setup file and in response, creates a new database record in the first database that is structured with the database structure for orders and also generates a new order identification that is unique to the system. The identification (e.g., a number) is stored for or in that newly created record in the first database.

At step 614, the second database server implements a monitoring application (a software application that is implemented to snoop, spy, or view activity in another application or database such as be reading data record locations) on the first database server or first data and uses the monitoring application to detect new activity in the Oracle database. At step 616, the monitoring application copies data values in the first database and provides them to the SQL database server in association with field and/or identification information. At step 618, the test equipment, in response to automatically reading an identification of a vial (e.g., machine readable code on a vial), reads or receives values from the first database that specify the operations (tests) that are to be performed as part of the order that is associated with that identification. The test information is transmitted and executed on the test equipment. The test equipment performs the tests and writes the resulting values generated from the testing to the first database (which in turn causes them to be copied and be live in the second database). Further, in operation, when there are multiple tests for a particular order, the generated test values are incrementally saved when the corresponding test is completed and in turn, is detected by the monitoring application and copied to the second database. In response, the web server and second database server change status in connection with that particular test and send a notification that some of the test results are now ready for live and current access.

FIG. 1C illustrates an overview of the process of ordering and managing blood samples according to an embodiment of the present invention. In particular, the arrows represent the communication pathways between various components of the system. To initiate the process, medical staff orders a blood sample for a patient using a physician client computing device 120. Via the portal 122, the medical staff places an order 125. The order 125 is transmitted to the patient database computer 200 and database 220 through the network and the web server.

The patient database computer 200 processes and stores the order 125. In particular, the order 125 is provided with a unique identification or accession number 125′, and is stored in the database 220 as a record under the accession number 125′. The stored order includes the patient information and the order parameters, e.g., what tests to perform on the patient's blood sample, directives for each test, instructions for interpreting the test results, etc. The stored order also includes a placeholder record or data field for the test results to be received from the lab at the completion of the blood tests. Further the patient database computer 200 transmits notice of the order 125 to the lab database 420 through the translator 300. A corresponding record under the accession number of the order is created in the lab database 420 to hold the test directive for the order. The lab database record also comprises data fields serving as a place holder for raw test data to be received from the test equipment.

After the order is stored in the patient database, elements of the order 125 are transmitted back to the physician portal 120 including the accession number 125′ to display an order confirmation for the physician. In some embodiments, the accession number 125′ is the order number.

Further, the web server transmits a notification to the patient portal application 132 on the patient client device 130 to inform the patient of the order. The notification is also provided on the web-based patient portal 132 delivered by the web server to the patient client device 130. The patient may review the order 125 and select a time and place where the patient is available for an appointment with a phlebotomist. The patient's selection is transmitted from the patient device 130 to web server.

The web server also transmits a notification to the phlebotomist portal application on the phlebotomist client devices of the pool of available phlebotomists in the patient's geographical area to inform them of the order 125. On their portals, the phlebotomists of the pool can view a list of available orders, including the time and place where each patient is available for a blood sample appointment. When a phlebotomist selects the patient to take the order, an appointment is made at the time and place provided by the patient.

The appointment triggers the shipment of sample vials bearing the patient's accession number 125′ to the phlebotomist. In some embodiments, the phlebotomist may print out machine-readable labels comprising the accession number to affix onto blank sample vials. The appointment also enables a communication function in the patient and phlebotomist portals that allows them to communication by chat, voice, or video. In some embodiments, the communication function of the portals is only enabled within specific a time window from the appointment (e.g., one hour before) to facilitate the coordination of the appointment. The phlebotomist portal is further provided with mapping and navigation features to assist with locating and accessing the site of the appointment.

After the blood sample appointment, the blood sample vials are scanned and shipped to the lab for testing. At the lab, the vials are loaded into the test equipment. The test equipment scans the labels for the accession number 125′, and for each accession number 125′, the test equipment retrieves the test directives previously stored in the record corresponding to that accession number in the lab database 420. The test equipment performs the test(s) on vials according to their respective test directives. At the completion of testing, the test equipment transmits the raw test data for the vials to the corresponding lab database record.

The translator 300 continuously monitors the content of the lab database 420 for new data. When a change of value in the placeholder data fields of the lab database is recorded, signaling the entry of new raw data from the test equipment, the translator 300 copies the raw data and saves it to the patient database 420. In some embodiments, the translator 300 generates a test report 128 from the raw test data of an order 125 according to the test parameters of the order 125. Unlike the raw test data, the test report 128 is a file or document formatted for human readability. In addition to presenting the test results, e.g., the amount of tested for substances detected in the patient's blood, the test report 128 for an order 125 may also interpret and provide context for the results in accordance with the test parameters of the order 125. After the lab database 220 receives the test result, the web server transmits the test report 128 to the patient portal 132 and physician portal 122 on their respective client devices 130, 120.

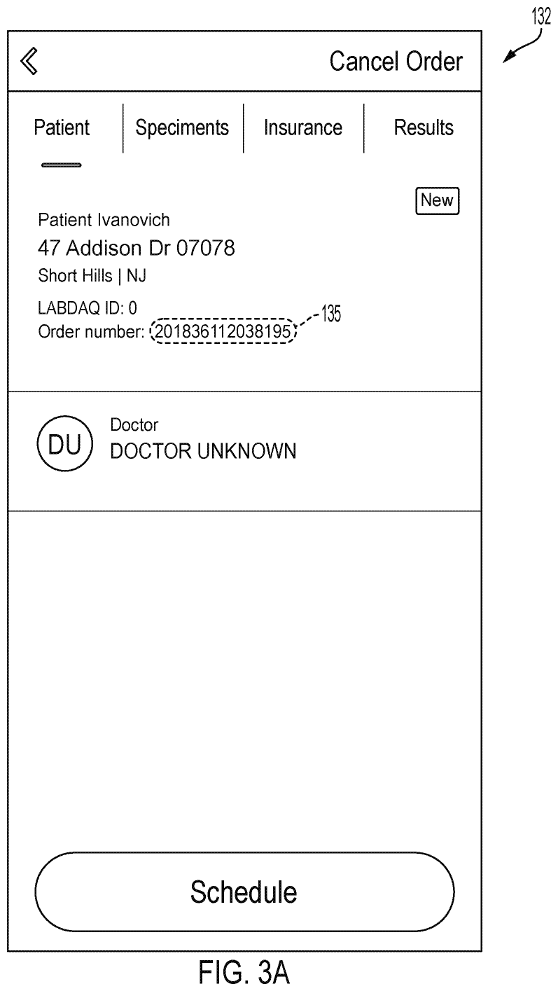

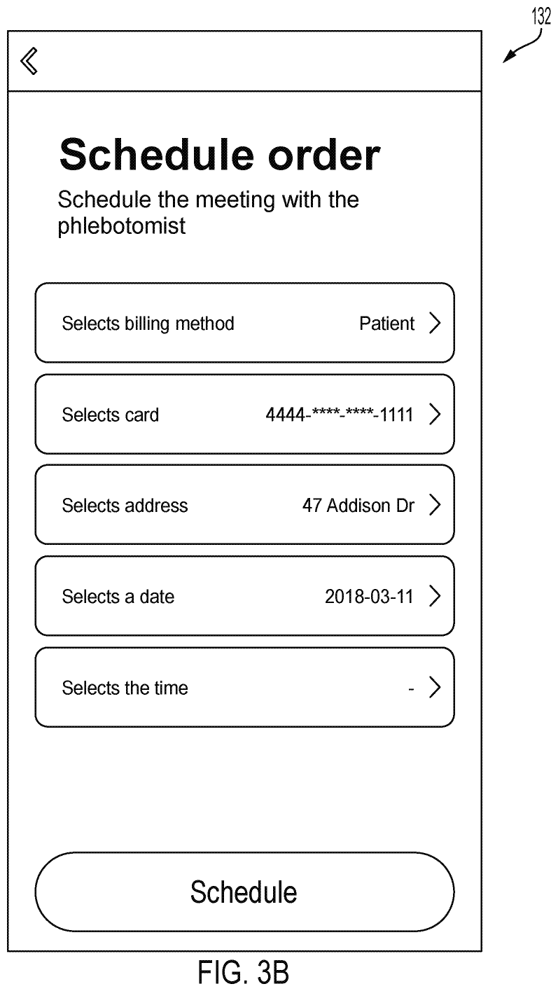

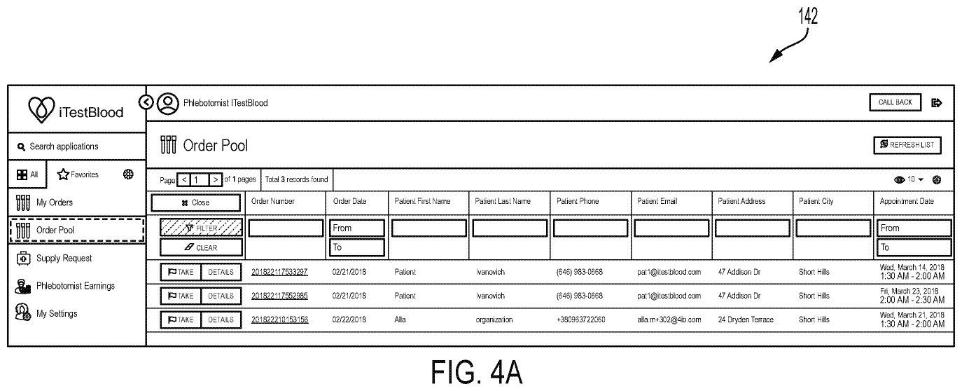

FIG. 3A illustrates an exemplary screenshot of a patient computing device displaying the order for blood sample analysis on the patient portal 132. As discussed above, the order was transmitted from the physician client device 130 to be processed by the patient database computer 200 via the web server 110 and network 115. An accession number 125′ (unique identification number) is issued for the order and the order is then made available to the patient computing device 130. The patient portal 132 further includes options for viewing a list of orders for blood samples, user setting to alter operational parameters of the portal, patient information/details, billing information, and other options pertinent to the patient.

As further illustrated in FIG. 3A, the patient is also provided with the means of choosing a phlebotomist and scheduling an appointment through the patient portal 122 on the client computing device 130. FIG. 3B illustrates another screenshot of the client computing device displaying the portal 122 for scheduling an order for a blood appointment with a phlebotomist. The patient may also change billing information and other data relevant to the transaction on this screen. For the blood sample appointment, the patient may select a date and time when they are available to meet a phlebotomist. In some embodiments, the patient selects a time window for the appointment. In some embodiments, the patient application/portal may detect the patient's location. Alternatively, the patient may enter a location where they desire the phlebotomist appointment to take place. After the patient selects a location and time on the portal 132 for the appointment, the patient's choice is transmitted from the patient client device 130 to the patient database computer 200 via the network 115 and through the web server 110. The patient database computer 200 records the selection and determines the phlebotomists available in that area from the pool of phlebotomists on file. The geographical area may be defined geometrically, e.g., within a radius around the location of the patient. The geographical area may also defined by city, county, region, or other geographical units relevant to the location.

A notification of the patient's availability for a blood sample appointment is broadcast to all registered phlebotomists within the geographical area. The notification is transmitted to phlebotomist client devices 140 by the web server 110 upon receiving the patient's choice of a time and place for the appointment. As with the physician and the patient, a phlebotomist portal 142 may be accessed with the phlebotomist client device 140. The client device may have a web browser configured to access a web portal 142 delivered by the server 110. The client device may also have a dedicated application in communication with the web server 110 and the database 220 over the network 115 and configured to display the phlebotomist's portal 142. The phlebotomist's portal 142 allows the phlebotomist to receive, schedule, and manage orders for blood samples.

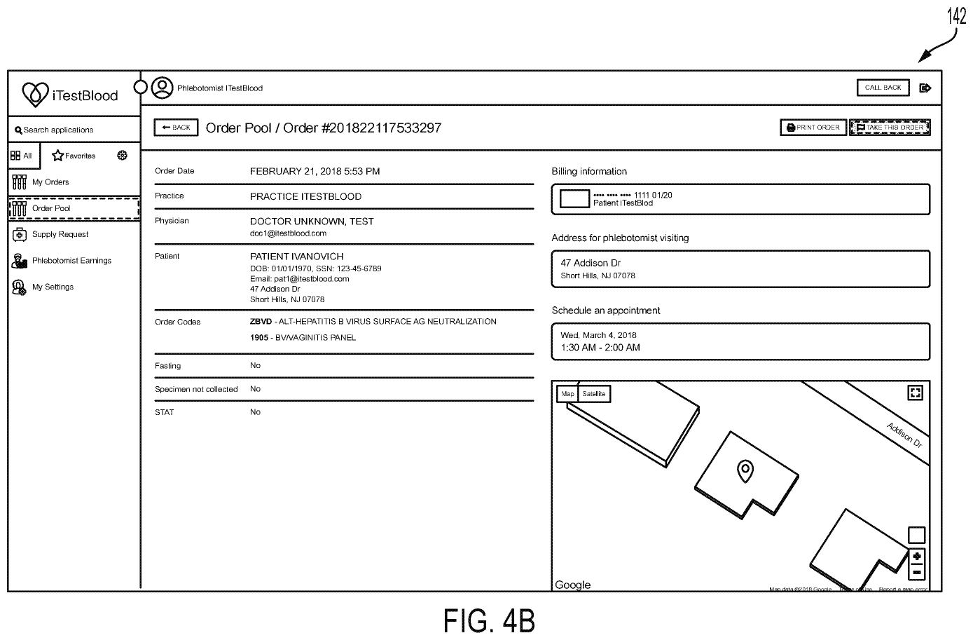

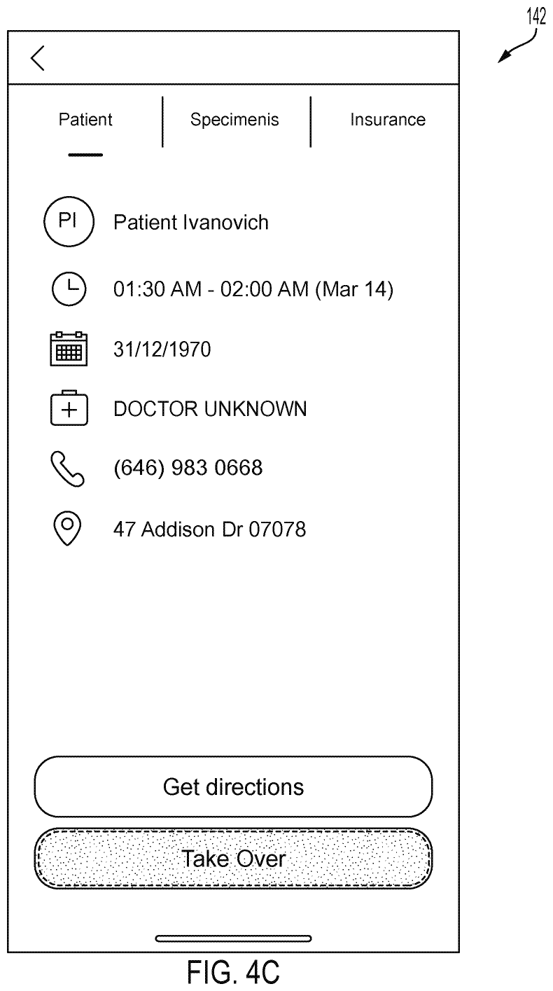

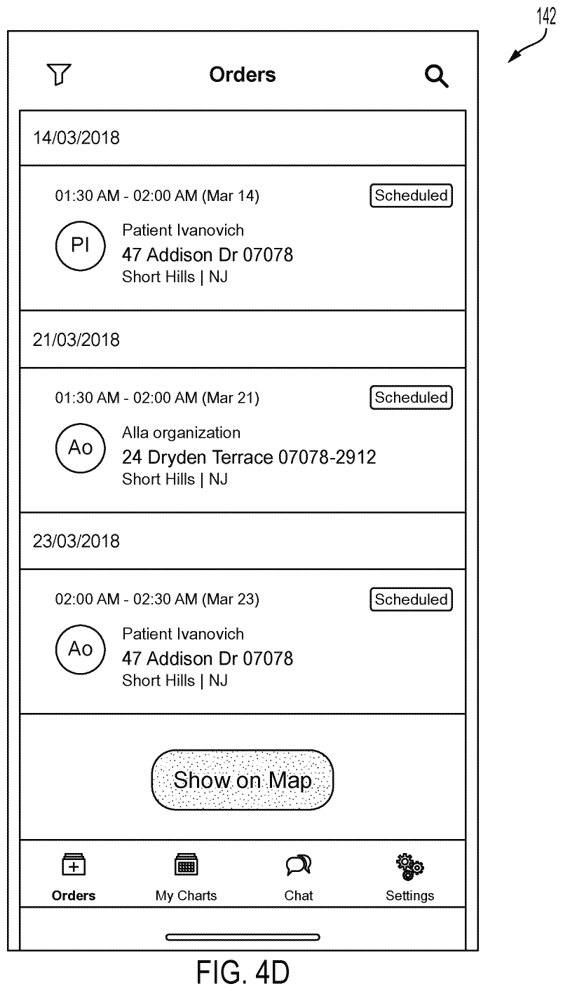

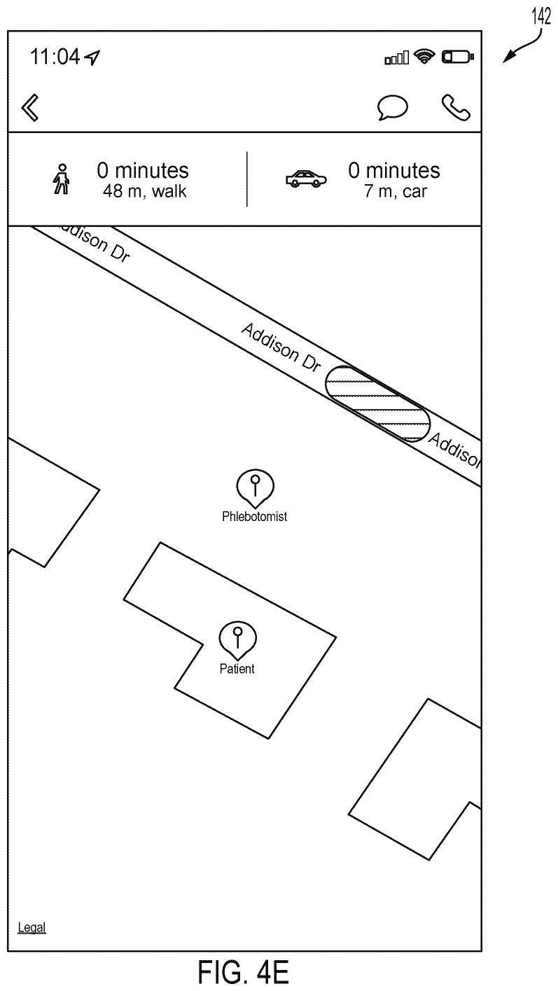

FIG. 4A illustrates a screenshot of an exemplary phlebotomist's portal 142 according to an embodiment of the principles disclosed herein. In particular, a list of orders available for the phlebotomist to select—or order pool, is provided. The listing for each patient on the portal 142 includes an order number, order date, and patient's name, address, and contact information. The portal 142 may provide other potentially useful information such as patient's distance from the phlebotomist. The list of orders thus represents the list of patients in the coverage area of the phlebotomists and for whom one or more orders for blood pending fulfillment test was placed and validated by the physician client device 120. The phlebotomist may select a particular order to view its details. FIG. 4B illustrates an exemplary screenshot of an order details screen on the phlebotomist's web portal 142. FIG. 4C illustrates another exemplary screenshot of an order detail screen on the phlebotomist mobile portal application 142. The order details include additional information on the patient, the order codes that specify the tests to be performed, and order parameters such as whether the blood should be taken while the patient has fasted. The order details screen further displays the time window where the patient is available for a blood sample appointment. The order details screen may also display a map showing the physical location of the client's address. A button is provided on the order detail screen for taking or accepting the order for blood sample. The order can also be accepted on the previously discussed order list screen illustrated in FIG. 4A where for each patient listed, the portal 142 also provides the option of selecting the patient for a blood test, or taking the order. The phlebotomist may take the order by clicking on the “take” button next to an order. The phlebotomist may accept and schedule several orders and embodiments of the phlebotomist portal 142 provide management tools for scheduling multiple order and for preventing or minimizing scheduling conflicts. FIG. 4D illustrates a screenshot of the phlebotomist portal 142 showing a list of scheduled orders on a phlebotomist mobile device 140.

When the phlebotomist takes an order (by clicking on the “take this order” button either on the detail page of an order or adjacent to the order in the list of orders page), thus making the appointment with the patient via the portal, the choice is transmitted from the phlebotomist client device 142 to the patient database computer 200 via the network 115 and web server 110. The web server 110 in turn notifies the client computer device 132 of the appointment.

In some embodiments, the patient database computer 200 may provide the phlebotomist's portal 142 with the accession number 125′ of the patient. The accession number 125′ may be provided on the portal as a file for a printable label to be affixed on a test vial. The label thus serves to identify and track the test vial carrying the patient's blood sample. The label may comprise a barcode, QR code, or other machine-readable visual representation of the accession number. The phlebotomist may print the label and attach it to a test vial to be used at the appointment with the patient.

In some embodiments, the system may cause tests vials carrying identifying labels to be shipped from the lab when the appointment is made. In this case, when the patient database computer 220 receives the acceptance of an order and notice of an appointment from a phlebotomist client device, in addition to notifying the patient client device, patient database computer may further transmit notice of the appointment to the lab database computer 400 via the translator 300. The lab database computer 400 may cause the lab to issue a vial bearing the accession number on a label as a barcode, QR code, or other machine readable visual identifier. Various levels of automation are possible for the generation of labeled vials. In some embodiments, the label may be printed out at the lab and attached to the vial by a human operator. In some embodiments, the lab database computer may automatically send instruction to a printer to print the labels for a specific order. The label may then get manually affixed to the dial. In other embodiments, the barcode, QR core, or other machine readable identifier may be printed onto a label already present on the vial in a fully automated process. The vial is then shipped to the phlebotomist for the blood sample appointment.

FIG. 5 illustrates the tracking of a sample vial 510 by the system 100. The label 520 allows the system 100 to track the physical location of the vial 510 is it travels from the lab to the phlebotomist and back. For example, a departure scan may be performed at the lab and registered before shipping the vial 510 to the phlebotomist. A notification of this departure scan may be sent to the phlebotomist's portal and/or computing device 140. The system may be configured to notify the physician portal 122 and client device 122 of the scan as well. Upon receipt of the vial, the phlebotomist may scan the vial using a scanner in communication with the phlebotomist portal via the phlebotomist client device 140. For example, the scanner may be connected to the phlebotomist with a cable, e.g., a USB cable. In some embodiments, the phlebotomist device 140 may comprise a scanner. For example, the phlebotomist client device 140 may be portable wireless device such as a tablet or a cell phone comprising a camera that can scan QR codes or read barcodes, as can many commercially available portable devices. In some embodiments, the portal application 142 on the phlebotomist client device 140 may operate the camera or scanner. The phlebotomist may perform an arrival scan when the vial is received, and a departure scan after the blood sample is taken and before the vial is shipped. When the vial 510 arrives at the lab, an arrival scan may be performed manually or automatically at the lab to register the arrival of the vial 510. The scans for each vial 510 are logged by the system 100 and transmitted to various portals and client devices depending on the system's configuration.

The system 100 may provide means of communication and navigation to users. For example, on the phlebotomist application or portal 142, a means of communication may be provided with the patient client device 130 when an appointment has been made between them. Communication may be established via a messenger, telephone, or video function in the patient and phlebotomist portal/applications 132, 142. In some embodiments, communication is only enabled a short time before the appointment, e.g., an hour. In such embodiments, the purpose of the communication means is to facilitate the coordination between the patient and the phlebotomist for the appointment. On phlebotomist client devices 140 that are portable, such as tablets or cell phones, the phlebotomist application or portal 142 may also feature a map and/or a navigation system for guiding the phlebotomist to the client's location for the blood sample appointment, in some embodiments. FIG. 4E illustrates a screenshot of map and/or navigation feature on a phlebotomist portal 142. In further embodiments, the application 142 may require the phlebotomist to check in on site, i.e., to explicitly signal his or her presence at the appointment location. Alternatively, check in may be accomplished automatically once the application 142 detects its location at the appointment site using geolocation features on the phlebotomist device 142. The check in is transmitted to the web server 110, which may then send a notification to the patient portal/device that the phlebotomist has arrived at the appointment site.

After the blood sample appointment between the patient and the phlebotomist, the phlebotomist scans and ships the vial 510 back. In a typical embodiment, the vial 510 may be placed on a tray with other vials and loaded into a test equipment configured to receive multiple vials 510 at once. The test equipment 500 scans the vials 510 and retrieves the test directive corresponding to each vial 510 from the lab database computer 400. The test equipment 500 then loads the vial 510 into the appropriate equipment for that vial, and performs the test according to the test directives. The appropriate equipment for each vial 510 is selected according to the test directives received from the lab database computer 400 for the corresponding accession number 125′. For example, the test directives may specify a centrifuge for a particular vial 510. The test directives may further specify how long the centrifuge should spin, or how many rotation, and at what speed, for example. The test directives are generated by the lab computer based on the test parameters included in the order.

In embodiments of the present invention, the equipment 500 is fully integrated with the system 100 and no human intermediary is required in the signal chain between the physician client device 140 and the test equipment 500 to provide instructions to the latter. Similarly, no human intervention is required in the opposite direction to transmit the test results from test equipment to the physician. The test equipment 500 receives testing directives from the lab database computer 400 and performs the test according to the directives. When a blood test for a vial 510 is completed, the test equipment 500 transmits raw test data to the lab database computer 400, along with the identifying information for the vial 510, i.e., the accession number 125′. The lab database computer 400 saves the raw test data in the empty cell of the placeholder record for the corresponding accession number 125′ that was created by the translator 300 when the order for blood sample was received. The raw test data may be stored in a standard format, or proprietary format, consistent or compatible with the rest of the system 100. To prevent data corruption and preserve the integrity of test data, the lab database 420 is configured to accept test data only from the test equipment 500. Further, the lab computer 400 and its database 420 represent the only connection that the test equipment has to the rest of the system 100. Specifically, the test equipment 500 has no communication link to the translator 300, the patient computer system 200 and database 220, the web server 110 or any of the client computing devices in communication with the system such as the physician 120, patient 130, or phlebotomist 140 client computing devices. None of these devices is able to interact with the test equipment 500. Only the lab computer 400 can communicate with the test equipment 500, specifically to provide test directives to the equipment 500 and receive raw test data therefrom. Further, the lab computer 400 itself only communicates with the rest of the system 100 through the translator 300, which serves as a buffer between the lab and the physician, patient, and phlebotomist systems.

The raw test data stored in the lab computer database 400 is used to generate a test report for the physician and patient. To that end, the translator 300 is configured to monitor the database 420 for changes, and in particular, the entry of raw test data. Specifically, the translator performs read-only access periodically to the lab database 420 to detect changes in values in the placeholder records. In some embodiments, the translator 300 monitors the database in real time. In some embodiments, the translator 300 may prioritize polling recently created placeholder records as they may be more likely to receive new raw data from the test equipment 500. When a change in the value of raw data is detected in the placeholder record, thus signaling that the blood of the patient corresponding to that record was tested and the test equipment 500 transmitted the raw test data to the database, the translator 300 accesses and reads the newly stored raw test data to generate a test report.

To generate a test report, the translator 300 translates the raw test data into a human readable format that the physician and/or patient can understand. The generation of the test report may be accomplished based on directives accompanying the order placed by the physician (i.e., the order parameters). For example, if the patient's blood sample is tested for blood sugar, the raw test data transmitted by the test equipment 500 to the lab database 420 may specify that a particular range of blood sugar is considered normal in the context of the patient's affliction. The translator 300 may thus indicate in the test results whether the blood sugar is low, normal, high, or any other designation specified by the test directives, for example. The translator 300 may also be configured to interpret the raw test data and to identify abnormalities in the results. The translator 300 then transmits the results to the patient database computer 200 for storage and distribution to the physician portal 122 and/or patient portal 132 depending on the order parameters. The patient lab database computer 200 may format the result into a test report in accordance with various formats specified by the order parameters or system configuration. The test report is stored in the patient's database record and can be served by the web server 110 to the appropriate client devices over the network.

Upon receipt of the test result, the patient database computer 200 immediately issues a notification for the patient on the client portal 132 and/or to the patient's client computing device 130. The system 100 may also be configured to notify the physician client device 120 or portal 122 first, and to obtain approval from the physician device to notify the patient. This is to allow the medical staff to review results and provide additional context to the results if needed before the patient receives them. In a typical embodiment, the phlebotomist client device 140 and/or portal 142 are not provided notification of, or access to the test results. Rather, the phlebotomists' active involvement with the blood test process ends when the vials 510 containing blood samples are shipped to the lab after the appointment. Regarding the physician's ability to review the test results before authorizing their release to the patient client device and/or portal, it should be noted that the patient database computer 200 is configured to prohibit any write access to the test result by the web server 110 or any of the client computer devices. Instead, only test results received from the translator can be written into the patient database computer records. As a result, the physician client computer 120 or any client device such as the patient client device 130 cannot alter the test results stored in the database computer 200. This restriction of access serves to protect the test results from tampering and preserve the integrity of the blood sample process.

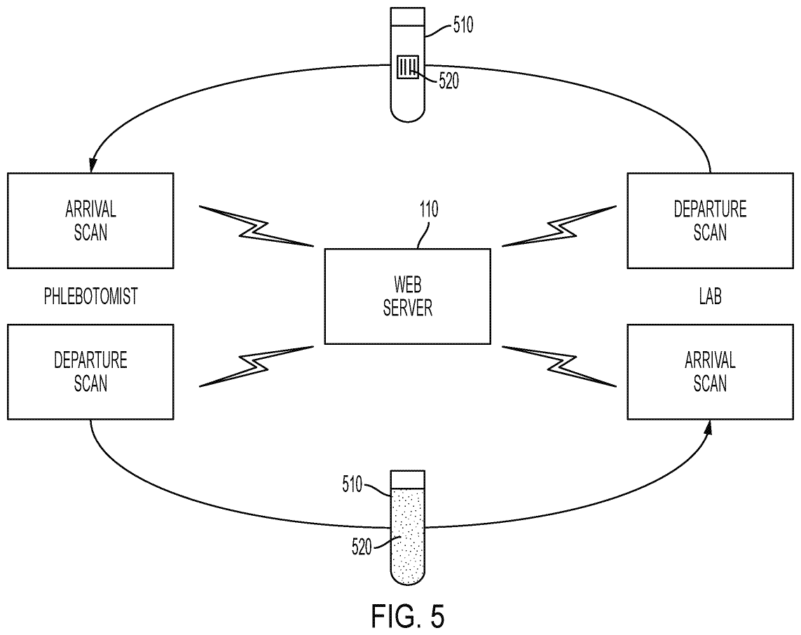

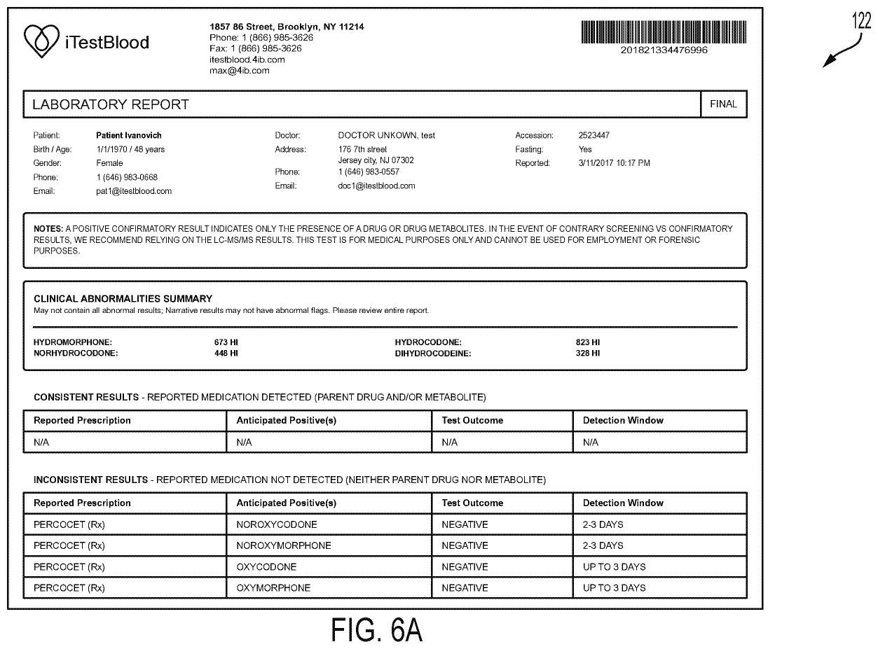

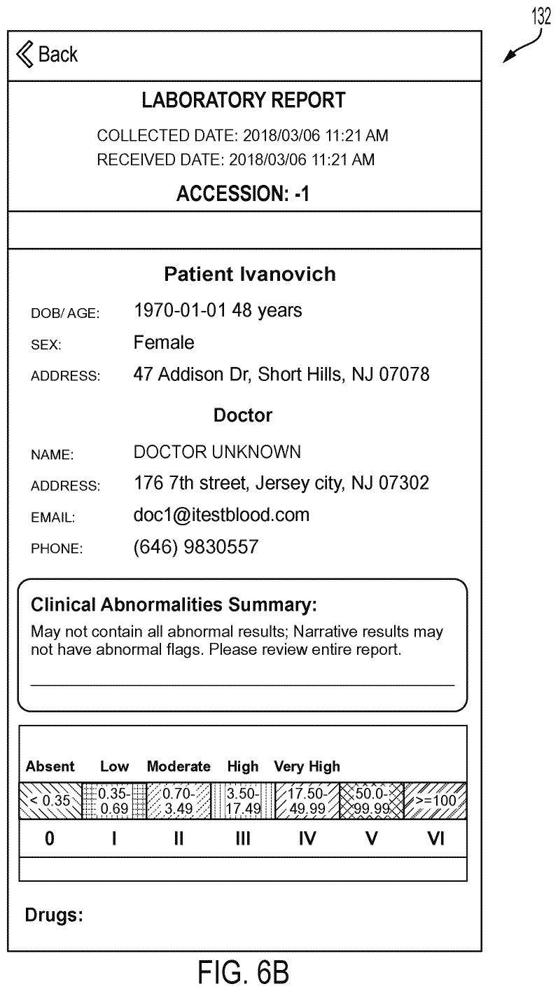

FIG. 6A illustrates a screenshot of an exemplary test report for the physician displayed the physician portal 122 on a physician client device 120. The test report features the client's biographical information as entered during the placement of the order. The test report is further identified by the accession number 125′ and corresponding bar code on the upper left-hand side of the report. The rest of the report provides and contextualizes the results from the test. For example, a summary of clinical abnormalities is provided and highlighted. The report also separates the results into results consistent with reported prescriptions and those that are not. It should be noted the analysis provided by the report is dependent on order parameters and system configuration. Accordingly, a test report for a different patient or for the same patient being tested for different elements may comprise different categories than those illustrated in FIG. 6A. FIG. 6B illustrates a screenshot of an exemplary test report for a patient displayed on the patient portal 132 on a mobile device 130. In addition to the patient information, the report provides a visualization of the test results using a color chart. Other visual tools may be used to present the test report on the patient or physician portals.

An includes feature of the system is that the translator or second database server implements a set of rules that reads the tests selected in a particular order and automatically determines the number of vials the phlebotomist will need to use for the order. For example, a physician's device may have ordered 5 blood tests and each may require a certain amount of blood to be drawn. The system using this features determine the number of vials needs for each of the 5 tests and transmits the total number of specific number for each test to the phlebotomist devices that is selected and is performing the blood draw. This provides an added layer of safety and efficiency by communicating at the order stage or at the shipping stage of the vials that a correct number was obtained.

The system is preferably implemented to include an application program interface (“API”). An application program interface is a set of defined commands (and defined responses to the commands) that are made available by a host computer (or software) and provide an automated communication channel between the host computer (or software) and a client computer (or software) that are automatically executed by the host computer (or software) and the client computer (or software) and further by the execution, make the functional features of the host computer (or software) automatically available to the client as a resource including the automatic execution of functional features by way of API commands and responses. The system can make the API available to customers such as hospital, which allows the customer to place a bulk or batch orders (e.g., for multiple tests at the same time or approximately at the same time) and to automatically update the status of each test on the customer's system using the APIs. This provides a highly efficient approach and involve the system automatically responding to orders and other commands from the customer or partner (e.g., transmit order, receive confirmation, receive status of order or individual tests in the order).

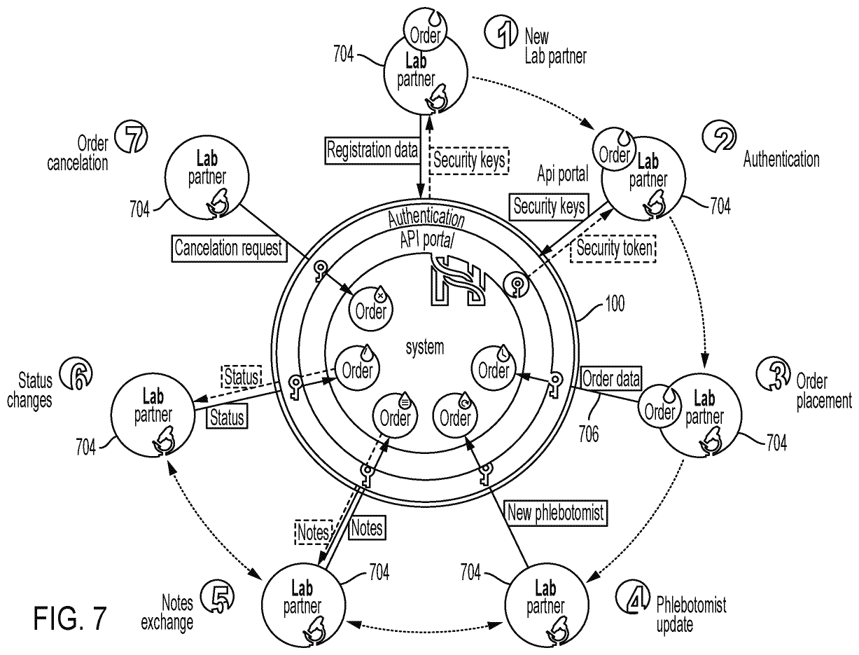

For example, with reference to FIG. 7, system 100 can be configured to incorporate an API comprising a set of defined (predefined) commands that are made available to customers or partners for automated computer to computer interface and communication. In FIG. 7, for illustration, a lab partner is identified as 704 and a process involving lab partner 704 (comprising the computer system of the lab partner) is shown in sequence around system 100. System 100 (e.g., using a web server 110, which is understood to include a server or computer that supports web server 110) is configured to implement the features and functionality illustratively depicted in FIG. 7. System 100 is configured to involve an initial security process involving a registration process wherein lab partner 704 registers with the system 100. In response to successful completion, system 100 transmits a set of security keys to the lab partner 704 that are specific to the lab partner 704. When lab partner 704 seeks to place an order (which can include many test requests), the lab partner 704 sends the security keys to the system 100, in response, system 100 (after checking the security keys) transmits a security token to the lab partner 704 for placing orders. The security token can be used in the subsequent communications (e.g., for a limited period of time) to authenticate communications by the lab partner 704. The lab partner 704 uses a specific API command for placing an order at step 706 which as a function of API would be automatically understood by the system 100 and automatically processed to create an order in the system 100 (and consequently, set the process in motion for having individual tests services and reported). This can include the assignment of a phlebotomist and reporting that information via a specific API command. The API commands include defined commands for transmitting notes, the status of an order or individual tests, and cancelling a test or order. In operation, the lab partner 704 has an existing internal software for its operations that can include the identification or ordering of phlebotomist tests and the API can be integrated into the internal software to allow the ordering process to be an interface option from within their internal software to send the order or test requests to the system 100 (to service and manage the test). The system 100 can handle the order automatically (without user intervention or involvement) including assigning a phlebotomist, processing a received test vial, conduct tests, and reporting on the results of the test. The API can be configured by system 100 to use HTTP commands such as GET and POST commands to communicate the commands between system 100 and lab partner 704.

Each of the systems, servers, computing devices, and computers described in this application is implemented on a computer system and can be configured to communicate over a network. A computer system may include a bus or other communication mechanism for communicating information, and a hardware processor coupled with bus for processing information. The computer system may also include a main memory, such as a random access memory (RAM) or other dynamic storage device, coupled to bus for storing information and instructions to be executed by processor. Main memory also may be used for storing temporary variables or other intermediate information during execution of instructions to be executed by processor. Such instructions, when stored in non-transitory storage media accessible to processor, render computer system into a special-purpose machine that is customized to perform the operations specified in the instructions. The computer system may further include a read only memory (ROM) or other static storage device coupled to bus for storing static information and instructions for processor. A storage device, such as a magnetic disk or optical disk, may be provided and coupled to bus for storing information and instructions. If desired, the computer system may be coupled via bus to a display, such as an LED monitor, for displaying information to a computer user. An input device, including alphanumeric and other keys, may be coupled to bus for communicating information and command selections to processor. Another type of user input device is cursor control, such as a mouse, a trackball, or cursor direction keys for communicating direction information and command selections to processor and for controlling cursor movement on display. This input device typically has two degrees of freedom in two axes, a first axis (e.g., x) and a second axis (e.g., y), that allows the device to specify positions in a plane. The computer system may implement the techniques described herein using customized hard-wired logic, one or more ASICs or FPGAs, firmware and/or program logic which in combination with the computer system causes or programs computer system to be a special-purpose machine. According to one embodiment, the techniques herein are performed by the computer system in response to the processor executing one or more sequences of one or more instructions contained in main memory. Such instructions may be read into main memory from another storage medium, such as storage device. Execution of the sequences of instructions contained in main memory causes the processor to perform the process steps described herein. In alternative embodiments, hard-wired circuitry may be used in place of or in combination with software instructions. It is understood to those of ordinary skill in the art that features or functionality illustratively described herein can be implemented using one or more sequences of computer-readable instructions that when executed provided the feature or functionality. The computer system is described in this paragraph in general purpose terms but this is not necessarily the case for each computer based devices described herein as is evident to those of ordinary skill in the art.

The term storage media as used herein refers to any non-transitory media that store data and/or instructions that cause a machine to operation in a specific fashion. Such storage media may comprise non-volatile media and/or volatile media. Non-volatile media includes, for example, optical or magnetic disks, such as storage device. Volatile media includes dynamic memory, such as main memory. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire and fiber optics, including the wires that comprise bus. Transmission media can also take the form of acoustic or light waves, such as those generated during radio-wave and infra-red data communications.

Various forms of media may be involved in carrying one or more sequences of one or more instructions to the processor for execution. For example, the instructions may initially be carried on a magnetic disk or solid state drive of a remote computer. The remote computer can load the instructions into its dynamic memory and send the instructions over a telephone line using a modem. A modem local to the computer system can receive the data on the telephone line and use an infra-red transmitter to convert the data to an infra-red signal. An infra-red detector can receive the data carried in the infra-red signal and appropriate circuitry can place the data on bus. Bus carries the data to main memory, from which processor retrieves and executes the instructions. The instructions received by main memory may optionally be stored on storage device either before or after execution by the processor.

The computer system may also include a communication interface coupled to bus. The communication interface provides a two-way data communication coupling to a network link that is connected to a local network. For example, the communication interface may be an integrated services digital network (ISDN) card, cable modem, satellite modem, or a modem to provide a data communication connection to a corresponding type of telephone line. As another example, the communication interface may be a local area network (LAN) card to provide a data communication connection to a compatible LAN. Wireless links may also be implemented. In any such implementation, the communication interface sends and receives electrical, electromagnetic or optical signals that carry digital data streams representing various types of information.

Network link typically provides data communication through one or more networks to other data devices. For instance, network link may provide a connection through local network to a host computer or to data equipment operated by an Internet Service Provider (ISP). ISP in turn provides data communication services through the worldwide packet data communication network now commonly referred to as the “Internet.” Local network and Internet both use electrical, electromagnetic or optical signals that carry digital data streams. The signals through the various networks and the signals on network link and through the communication interface, which carry the digital data to and from the computer system, are example forms of transmission media.

The computer system can send messages and receive data, including program code, through the network(s), network link and the communication interface. In the Internet example, a server might transmit a requested code for an application program through Internet, ISP, local network and the communication interface.

The code may be executed by the processor as it is received, and/or stored in storage device, or other non-volatile storage for later execution.

It should be understood that variations, clarifications, or modifications are contemplated. Applications of the technology to other fields are also contemplated.

Exemplary systems, devices, components, and methods are described for illustrative purposes. Further, since numerous modifications and changes will readily be apparent to those having ordinary skill in the art, it is not desired to limit the invention to the exact constructions as demonstrated in this disclosure. Accordingly, all suitable modifications and equivalents may be resorted to falling within the scope of the invention.

Thus, for example, any sequence(s) and/or temporal order of steps of various processes or methods that are described herein are illustrative and should not be interpreted as being restrictive. Accordingly, it should be understood that although steps of various processes or methods or connections or sequence of operations may be shown and described as being in a sequence or temporal order, but they are not necessarily limited to being carried out in any particular sequence or order. For example, the steps in such processes or methods generally may be carried out in various different sequences and orders, while still falling within the scope of the present invention. Moreover, in some discussions, it would be evident to those of ordinary skill in the art that a subsequent action, process, or feature is in response to an earlier action, process, or feature.

It is also implicit and understood that the applications or systems illustratively described herein provide computer-implemented functionality that automatically performs a process or process steps unless the description explicitly describes user intervention or manual operation.

It is understood from the above description that the functionality and features of the systems, devices, components, or methods of embodiments of the present invention include generating and sending signals to accomplish the actions.

It should be understood that claims that include fewer limitations, broader claims, such as claims without requiring a certain feature or process step in the appended claim or in the specification, clarifications to the claim elements, different combinations, and alternative implementations based on the specification, or different uses, are also contemplated by the embodiments of the present invention

It should be understood that combinations of described features or steps are contemplated even if they are not described directly together or not in the same context.

The terms or words that are used herein are directed to those of ordinary skill in the art in this field of technology and the meaning of those terms or words will be understood from terminology used in that field or can be reasonably interpreted based on the plain English meaning of the words in conjunction with knowledge in this field of technology. This includes an understanding of implicit features that for example may involve multiple possibilities, but to a person of ordinary skill in the art a reasonable or primary understanding or meaning is understood.

The words “may” or “can” express that are typically used to communicate that variations are contemplated. This is just a precaution that is sometimes used.

It should be understood that the above-described examples are merely illustrative of some of the many specific examples that represent the principles described herein. Clearly, those skilled in the art can readily devise numerous other arrangements without departing from the scope as defined by the following claims.

1. A system for securely providing and processing biological specimen analysis, the system comprising:
a web server configured to receive an order for a biological specimen analysis from a provider client computer;
a patient database computer in communication with the web server and patient database, and configured to receive the order and generate order parameter data and a unique ID associated with the order, and further configured to store data comprising the order parameter data, the unique order ID, and test results for each order in the patient database as a patient database record;
lab test equipment configured to analyze biological specimens and transmit raw test data;
a lab database computer in communication with the lab test equipment and comprising a lab database configured to store the raw test data received from the lab test equipment only; and
a translator computer in communication with both the patient database computer and the lab database computer and configured to detect storage of the order in the patient database, and further configured to communicate with the lab database computer to create a placeholder record for the order in the lab database, the placeholder record being associated with the unique order ID and comprising the order parameter data, and further comprising cells for storing the raw test data received from the lab test equipment and associated with the order.

2. The system of claim 1 wherein the translator computer communicates with the lab database computer to monitor the lab database for receipt of the raw test data transmitted by the lab test equipment, and wherein when the translator computer detects the receipt of the raw test data, the translator computer retrieves the raw test data from the lab database, performs an analysis on the raw test data to generate test results, and transmit the test result to the patient database computer for storage in the patient database record of the unique order ID.

3. The system of claim 1 wherein the patient database computer is further configured, in response to receiving an order, to transmit a notification of an available order to a pool of phlebotomist devices based on location or other criteria, designate a phlebotomist device to be matched to the order in response to that device accepting the order, track the phlebotomist device within a predetermined time period before the appointment, and coordinate communications between the phlebotomist and the patient.

4. The system of claim 1, wherein the analysis performed by the translator computer is performed according to the order parameters associated with the unique order ID and comprises translating the raw test data to a human readable format.

5. The system of claim 4, wherein the analysis performed by the translator computer detects abnormal results and provides a diagnostic.

6. The system of claim 1, further comprising a test vial for storing a specimen, the test vial comprising the unique order ID and a barcode for the unique order ID.

7. The system of claim 1, wherein the lab test equipment comprises a scanner configured to scan the unique order ID on the test vial and to associate the raw test data generated from testing the test vial with the unique order ID of the test vial for transmission to the database computer.

8. The system of claim 1, wherein the translator computer monitors the lab database in real time.

9. The system of claim 1, further comprising a phlebotomist computer device in communication with the web server and configured to receive the order and the unique order ID from the web server, wherein the web serve retrieves the order and the unique order ID from the patient database, the phlebotomist computer device being further configured to enable printing of the unique order ID as a label comprising the unique order ID and a barcode for the unique order ID.

10. The system of claim 1, further comprising a patient computer device in communication with the web server and configured to receive the test results from the web server, wherein the web server retrieves the test results from the patient database.

11. The system of claim 1, wherein the order parameter data comprises at least one of instructions to perform a specific test and instructions for translating the raw test data.

12. The system of claim 9, wherein the lab test equipment retrieves the order parameter data from the database computer and performs testing according to the order parameter data.
